(19) United States
(12) Patent Application Publication
(10) Pub. No.: US 2023/0023786 A1
(43) Pub. Date: Jan. 26, 2023

(54) METHODS OF MODIFYING MICRONEEDLES AND NEEDLES FOR TRANSDERMAL ELECTROCHEMICAL DETECTION OF IONS AND (BIO)MOLECULES

(71) Applicants: Gaston Adrian CRESPO PARAVANO, Stockholm (SE); Maria CUARTERO BOTIA, Stockholm (SE)

(72) Inventors: Gaston Adrian CRESPO PARAVANO, Stockholm (SE); Maria CUARTERO BOTIA, Stockholm (SE); Marc PARRILLA PONS, Stockholm (SE)

(21) Appl. No.: 17/784,188

(22) PCT Filed: Dec. 1, 2020
(86) PCT No.: PCT/SE2020/051154

§ 371 Date: Jun. 10, 2022

(30) Foreign Application Priority Data
Dec. 11, 2019 (SE) 1951427-2

(51) Int. Cl. B05D 7/00; A61B 5/145; A61B 5/1473; A61B 5/00; B05D 7/16

(52) U.S. Cl. CPC B05D 7/544; A61B 5/14514; A61B 5/14546; A61B 5/1473; A61B 5/6833; A61B 5/685; B05D 7/16

(57) ABSTRACT

The disclosure relates to two methods to modify microneedles and needles to transform them as electrochemical sensors for ions and biomolecules. The methods focus on microneedles and needles made of any material through an external and internal modification methods to provide the function as electrodes: the working electrode, (pseudo)counter electrode and/or (pseudo)reference electrode depending on the electrochemical readout. With the external modification method, any solid microneedle and needle can be individually transformed in either of the said electrodes. With the internal modification method, any hollow microneedle and needle can be individually transformed in either of the electrodes. The working electrode, (pseudo)counter electrode and or (pseudo)reference electrode can be simultaneously integrated into the same hollow microneedle or needle by internal compartmentation. Two different biofluids can be simultaneously targeted by microneedles and needles of different sizes, structures and fabricated by one or both methods when integrated in the same skin patch.

### TECHNICAL FIELD

The present disclosure relates to methods of modifying microneedles and needles, in particular to be applied in electrochemical devices with medical and sport performance purposes. More specifically, the disclosure refers to on-body wearable patches for ion and (bio)molecule transdermal detection in a real time way and simultaneously in two different biofluids.

### BACKGROUND

On-body wearable devices are the current core for modern clinical analysis and sport performance monitoring. Such devices are able to digitalize physical and chemical events into information useful for the early diagnosis, evaluation and monitoring of health diseases as well as the assessment of the body status of athletes. Thus, the devices commonly comprise the sensory part for the analysis of certain chemical composition, electronics and display elements. The present invention focuses on the sensory part dedicated to the electrochemical detection of ions and (bio)molecules. The main biological fluids in where these chemicals are determined are sweat, urine, interstitial fluid and blood. In particular, two options are in principle plausible for the on-body analysis of interstitial fluid and blood:

(i) The fluid is extracted out of the skin of the individual and such extraction is coupled to the sensory element; and (ii) the sensory element is implanted inside the skin of the individual.

Both approaches imply certain level of invasiveness and one common way to achieve them is through the use of needles. Implantable sensors are in principle preferable for truly in-situ and real time measurements without any kind of sample manipulation. Thus, the present disclosure relates to the implementation of sensory elements in microneedle and needle devices structured for transdermal detection of ions and (bio)molecules in real time by means of electrochemical readout.

Below some documents will be briefly discussed being in the same the technical field as the present disclosure.

IN-201811041978 describes a process for making solid-state ion sensors for on-chip determination of potassium ion (K⁺) in body fluid. The application describes a potentiometric sensor based on microneedles for determination of K⁺. The microneedle serves as a sampling platform to bring the interstitial fluid to a sensor surface, which is placed outside the skin. The electrode does not act in a transdermal configuration, but it is coupled to the microneedle for sampling instead.

U.S. Pat. No. 9,743,870 relates to microneedle arrays for biosensing and drug delivery. A device is provided for detecting an analyte and/or releasing a biochemical drug into a biological fluid. The device can include an array of microneedles exclusively of hollow type, in which each needle includes a protruded structure including an exterior wall forming a hollow interior and an opening at a terminal end of the protruded needle structure that exposes the hollow interior, and a probe inside the exterior wall to interact with one or more chemical or biological substances that come in contact with the probe via the opening to produce a probe sensing signal. The patent focuses on the description of how to convert tips into electrochemical electrodes, said as the probe, that can be introduced into hollow microneedles to facilitate transdermal measurements. The microneedle is not modified in its nature and is hence acting as a holder for the sensor and the sample. The described method is based on the use of ‘pastes’ or ‘mixtures’ composed of the sensing elements to analyse biomolecules.

U.S. Pat. No. 9,182,368 discloses a method of manufacturing a sensor for sensing analytes wherein a microsensor comprises an array of filaments and the microsensor can be used for potentiometric measurement of e.g., K⁺. The patent relates to the external modification of microneedles exclusively of solid type. The microneedles are first fabricated to be entirely protected with an insulating coating and then a portion is peeled to expose the sharp end of the microneedle for further modification. This procedure is accomplished by means of a dicing saw. The first modification of the exposed sharp end of the microneedle is with a conductive layer and then with a series of sensing elements to provide different kind of sensors. All the sensing elements are physically attached to the external wall of the solid microneedle without the apparent use of any chemical procedure.

WO-2016018148 A1 describes a biosensor comprising a modified metal surface and the method for the modification of a metal surface exclusively selected from the group consisting of Ru, Rh, Pd, Ag, Ir, Pt and Au, and wherein an enzyme is covalently attached to the metal surface via an alkyloxy or an alkenyloxy moiety.

GB 2539224 A relates to a method for forming a chemical sensors device as well as the device, which is particularly devised for the simultaneous detection of pH and chemical concentration of the analyte(s) on the basis of a multi-electrode platform. The pH electrode is used to correct the response of the rest of electrodes, which are based on enzymes and therefore its response is in turn pH dependent. The method to fabricate the electrodes is proposed under two designs to provide either planar or cylindrical configuration. Both configurations are claimed to be suitable for both stand-alone devices, where the sample solution is deposited or conveyed by fluidic transport means to the sensing layers, and in implantable and skin patch-like sensor devices, where the device is applied for measuring the pH and the concentration of given substances in situ. WO 2019186129 A1 discloses a microneedle platform for sensing and delivery, and it concerns a dual function for a microneedle patch for delivering an agent and sensing an analyte typically associated with the delivery or effect of such agent. Also, it concerns the method of delivering the agent and sensing the analyte. Solid microneedles are designed to be coated with a dissolvable or swellable material that contains the agent to be delivered after an applied stimulus. The sensor in the solid microneedle is provided with functionalization; first with a conductive coating and then with an extra coating that is defined to react with the delivered analyte to produce a voltage/current or resistance/impedance. Alternatively, the agent delivery can be accomplished through the aperture of a hollow microneedle by means of a pump system. The same hollow microneedle is somehow modified to simultaneously provide the sensing method: the microneedle is fashioned to have a plurality of faces or sides such that a part thereof has at least one aperture therein, connected to an agent supply, and is used for agent delivery and another said faces or sides is solid and used for sensing purposes.

The object of the present invention is to achieve improved methods of fabrication of microneedles and needles serving as electrochemical sensors and structured for painless transdermal detection of ions and/or (bio)molecules in real time. Then, the microneedles and needles structured to act as sensors for different analytes are integrated in a patch that is suitable to be applied to the skin. Two different biofluids may be targeting simultaneously by microneedles and needles of different sizes, structures and designs integrated in the same skin patch.

### SUMMARY

The above-mentioned object is achieved by the present invention according to the independent claims.

Preferred embodiments are set forth in the dependent claims.

The present disclosure relates to two methods covering two different aspects of the present invention.

Thus, two methods for the fabrication of electrochemical sensors for ions and molecules, preferably biomolecules, in the form of microneedles and needles will be described. The methods focus on the modification of any available microneedles and needles, in terms of materials, nature and dimensions, that are externally (method 1) and/or internally (method 2) converted into electrochemical sensors.

Ion detection is preferably (but not restrictedly) achieved by means of potentiometry readout, requiring the manufacturing of the working and reference electrode. Molecule (biomolecule) detection is preferably (but not restrictedly) achieved by means of amperometry readout, requiring the manufacturing of the working electrode, pseudoreference electrode and pseudocounter electrode.

In both cases, variations can involve any kind of electrochemical readout (potentiometry, amperometry, voltammetry, chrono-techniques, coulomb-techniques and impedance among others). These electrodes can be implemented by any skilled person in the filled in a biocompatible polymeric substrate that allows for on-body portability: forming a sensing (micro)needle-based patch.

Both methods provide adequate final dimensions of the modified microneedles and needles for painless skin penetration. Both methods are based on a reduced number of steps for the modification of the microneedles and needles, simplifying the entire manufacturing procedure with respect to the state of the art and providing improved cost-effectiveness and further compatibility with mass production. Both methods can be applied to any (micro)needle of any material and length, respecting its ability to be inserted into the skin while providing the sensor element. With the different lengths, either interstitial fluid and/or blood can be reached for transdermal analysis. The combination of microneedles and needles in the same patch provides the simultaneous detection of the same or different analyte(s) in the two biological fluids.

The (micro)needle skin patch presents compatibility for multi-analyte detection and the gathering of measurement reproducibility. Both can be achieved by a combination of the different types of sensing microneedles and needles herein described.

For the method 1, depending on the microneedle or needle material, conductivity can be firstly increased with appropriate chemical or physical (i.e., coating) modification. Also, depending on the microneedle or needle material, a first modification to facilitate the chemical functionalization with further elements needed for the sensing event can be implemented. Then, a series of layers/elements are added to provide the sensing capacity to the microneedle or needle. All the electrodes obtained through the method 1 are to be fabricated in separate microneedles or needles.

For the method 2, hollow microneedles or needles are internally modified. This can be accomplished with a chemical and/or physical modification of the walls of the microneedles or by the fitting of a physically/chemically modified wire. The utilized elements are analogous to those presented in method 1. All the electrodes obtained through the method 2 can be fabricated in separate microneedles or needles. All the electrodes obtained through the method 2 can be fabricated in only one microneedle or needle by means of an internal modification of the microneedle or needle in different compartments to allocate each electrode. For example, for the potentiometric detection of an ion, the microneedle or needle contains two separate internal compartments: one for the working electrode and the other for the reference electrode. For the amperometric detection of a biomolecule, the microneedle or needle contains three separate internal compartments: one for the working electrode, the second one for the pseudocounter electrode and the last one for the pseudoreference electrode.

The following description is related to the preferred methods for the conversion of any microneedle and needle in wearable sensors for painless transdermal detection of ions and molecules in interstitial fluid and/or blood. Throughout the description it is referred to detection of molecules, that includes detection of biomolecules. The detection is to be accomplished electrochemically. It is described the fabrication of the working electrode and the reference electrode needed for ion detection by potentiometry. It is also described the fabrication of the working electrode, pseudoreference electrode and pseudocounter electrode needed for molecules' detection by amperometry. However, every electrochemical readout can be used with the fabricated microneedles and needles (e.g., potentiometry, amperometry, voltammetry, chrono-techniques, coulomb-techniques, and impedance, among others). In principle, any electrochemical sensor can be translated into a microneedle and/or needle-based sensor following either method 1 or 2 in this invention. While variations of the fabrication of the working electrode are admissible until the required dimensions of the tip of the needle to assure painless insertion, the reference electrode, pseduoreference electrode and pseudocounter electrode can be universally used.

In one embodiment, every electrode required for the electrochemical readout is implemented in individual microneedles and/or needles that are in turn implemented in a polymeric substrate, hence, to fabricate the wearable sensing patch. But all the described electrodes can be fabricated inside the same microneedle or needle owing to an internal modification to produce different compartments for each electrode. It is here also described the compatibility of one patch for multi-analyte detection, the gathering of measurement reproducibility or the simultaneous analysis of interstitial fluid and blood. This latter is accomplished by the simultaneous integration of microneedles and needles of different length, and may be dimensions, to reach two different biological fluids such as interstitial fluid (e.g., with the microneedles) and blood (e.g., with the needles). The (micro)needle patch can be fashioned in the way to provide several electrochemical readouts in the same patch.

The two methods for microneedle and needle modification into sensors are described for hand-made preparation in a cost-effective way by any person skilled in the regular fabrication of electrochemical sensors. But the present invention is also compatible with a mass production approach by the automatization of all the involved steps. Both methods for the preparation of the (micro)needle-based sensors prevent from the detachment of the layers, films and/or coatings utilized in each procedure before, during and after skin penetration. The integration of each electrode in a separate solid or hollow microneedle and/or needle but also the internal compartmentation of the microneedle and/or needle to host the sensing element allows to avoid any interference between the electrodes. For example, mixed potentials in potentiometry readout and cross talking between electrodes.

The wearable sensing (micro)needle patch is compatible with an electronics module that allows for the analyte sensing through fluctuations in the analytical signal (e.g., voltage, current, resistance, capacitance, impedance, etc.). The wearable sensing (micro)needle patch preferably detects parameters that are depending on the analyte concentration/activity/level in a biological fluid but also in any kind of sample. This dependence is achieved owing to the sensing element implemented in the microneedles and/or needles acting as the working electrode. The trend of such parameter with the analyte concentration/activity/level is preferably fitted to a mathematical equation acting as the calibration graph for each sensor-analyte tandem. The calibration graph is preferably recorded before on-body measurements with the wearable sensing microneedle patch reading the parameter of standard solutions of known concentration/activity/level of the analyte. The calibration graph is preferably linear, but it could be whatever mathematical equation that fits the measured parameter with the analyte concentration/activity/level. The calibration graph serves to calculate unknown concentrations of the analyte in the corresponding biological fluid. For this purpose, on-body measurements can be accomplished at discrete time points (e.g., every minute, hour and day) or in a continuous way. In one specific example, the wearable sensing (micro)needle patch is configured for the transdermal detection of ions (e.g., potassium, sodium, pH, magnesium, calcium, ammonium, chloride, etc.) through potentiometric readout by the fabrication of a working electrode and reference electrode. In other specific example, the wearable sensing (micro)needle patch is configured for the transdermal detection of molecules (e.g., glucose, lactate, alcohol, hormones, creatinine, etc.) through amperometric readout by the fabrication of a working electrode, pseudoreference electrode and pseudocounter electrode. In all the cases, the wearable sensing (micro)needle patch detects at least one analyte. In one variation, the wearable sensing microneedle patch combines more than one readout (e.g., potentiometry and amperometry). In another variation, the wearable sensing (micro)needle patch combines more than one length of the microneedles and/or needles to provide simultaneous detection in interstitial fluid and blood. It is also possible that the (micro)needle patch contains several microneedles and/or needles of the same type in terms of the working electrode to obtain replicates of the measurements aiming at an optimized accuracy of the analyte quantification as an average or all the replicates. Also, the replicates may help in distinguishing outlier measurements.

The present invention focuses on the transforming of any microneedle and/or needle into the electrodes necessary for electrochemical measurements. The working electrode and the reference electrode are needed for ion detection by potentiometric readout. The working electrode, the pseudoreference electrode and the pseudocounter electrode are needed for molecule detection by amperometric readout. The described methods for the fabrication of all the electrodes can be modified to detect any kind of analyte by any kind of electrochemical technique. The needed (micro)needle-based electrodes for sensing at least one analyte are incorporated in a polymeric substrate for on-body measurements. Such (micro)needle-based sensing patch can be adapted for any analyte(s) as desired, using a combination of different electrochemical readouts and comprising different microneedle length in order to reach both interstitial fluid and blood.

### BRIEF DESCRIPTION OF THE DRAWINGS

FIG. 1 is a schematic illustration related to method 1 regarding a working electrode in the form of microneedle or needle.

FIG. 2 is a schematic illustration related to method 1 regarding a reference electrode in the form of microneedle or needle.

FIG. 3 is a schematic illustration of applying microneedles of FIGS. 1 and 2 for transdermal ion detection in interstitial fluid.

FIG. 4A shows the response at increasing concentrations (calibration graph) for microneedles structured for the potentiometric detection of potassium ions in artificial interstitial fluid before and after skin penetration using a microneedle patch.

FIG. 4B shows the response at increasing concentrations for potassium ions before and during skin penetration.

FIG. 4C shows the response of chicken skin pieces conditioned at a fixed pH and the calibration graph of the pH microneedle sensor.

FIG. 5 is a schematic illustration related to method 1 regarding a working electrode in the form of microneedle or needle.

FIG. 6 is a schematic illustration related to method 1 regarding a pseudoreference electrode in the form of microneedle or needle.

FIG. 7 is a schematic illustration related to method 1 regarding a pseudocounter electrode in the form of microneedle or needle.

FIG. 8 is a schematic illustration of applying microneedles of FIGS. 5-7 for transdermal molecule detection in interstitial fluid.

FIG. 9 is a calibration graph for glucose using microneedles.

FIG. 10 illustrates a patch comprising microneedles and/or needles modified for ion and molecule detection.

FIG. 11 is a schematic illustration related to method 2 regarding a working electrode in the form of microneedle or needle.

FIG. 12 is a schematic illustration related to method 2 regarding a reference electrode in the form of microneedle or needle.

FIG. 13 is a schematic illustration related to method 2 showing a cross-sectional view of a microneedle or needle with two compartments.

FIG. 14 is a schematic illustration related to method 2 regarding a working electrode comprising a sensor wire.

FIG. 15 is a schematic illustration related to method 2 regarding a reference electrode comprising a sensor wire.

FIG. 16 is a schematic illustration related to method 2 showing a cross-sectional view of a microneedle or needle with three compartments.

FIG. 17 discloses a calibration graph for potassium ions in artificial interstitial fluid.

FIG. 18 is a schematic illustration of a patch provided with microneedles and/or needles modified according to method 2 of the present invention.

### DETAILED DESCRIPTION

The two methods, and the resulting (micro)needle patches, will now be described in detail with references to the appended figures. Throughout the figures, the same, or similar, items have the same reference signs. Moreover, the items and the figures are not necessarily to scale, emphasis instead being placed upon illustrating the principles of the invention.

In the following description references within parenthesis refer to steps in the figures, and sometimes also to the resulting microneedle or needle illustrated in that step.

Method 1 and method 2 will be described separately. Each of these descriptions will start with a more general depiction of the respective method and will then be followed by a more detailed explanation that includes numerous examples.

### Method 1— External Modification of Solid Microneedles and Needles

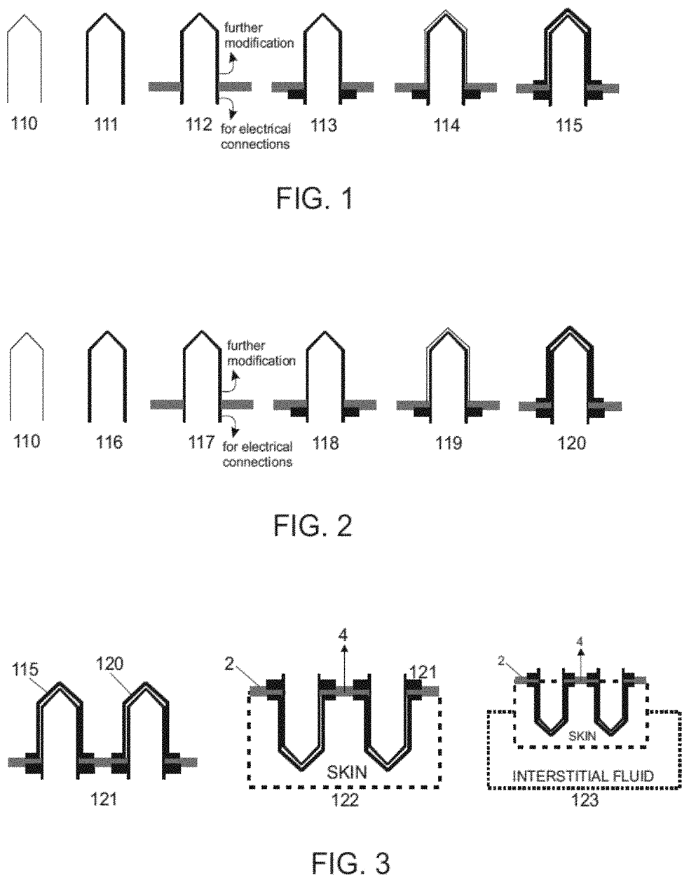

The present invention relates to a method, method 1, of modifying the external surfaces of at least two solid microneedles or needles to be arranged in a (micro)needle-based patch configured for ion and/or molecule on-body transdermal sensing. Method 1 will be described with references to FIGS. 1-10.

The method comprises providing at least one microneedle or needle (110) structured to function as a working electrode (115, 126) for ion sensing or molecule sensing. Depending on the material of the microneedle or needle, this will be first modified to improve its conductivity and/or to provide a material compatible with a further chemical modification. The microneedle or needle can be directly modified with the sensing elements. For example, a stainless-steel microneedle or needle requires for a coating to improve the conductivity before adding the sensing element. A glassy carbon microneedle or needle can be chemically modified with appropriately designed monolayers to provide the sensing element. The method comprises attaching the at least one microneedle to a substrate of said (micro)needle-based patch (see FIGS. 1 and 5).

The method further comprises providing at least one microneedle or needle (110) structured to function as a reference electrode (120) for ion sensing, or as a pseudoreference electrode (127) for molecule sensing, coating said at least one microneedle or needle with an Ag/AgCl layer coating to improve the conductivity of the microneedle or needle and serving as the basis of a reference electrode or pseudoreference electrode, and then attaching the at least one microneedle or needle to the substrate of said (micro)needle-based patch (see FIGS. 2 and 6).

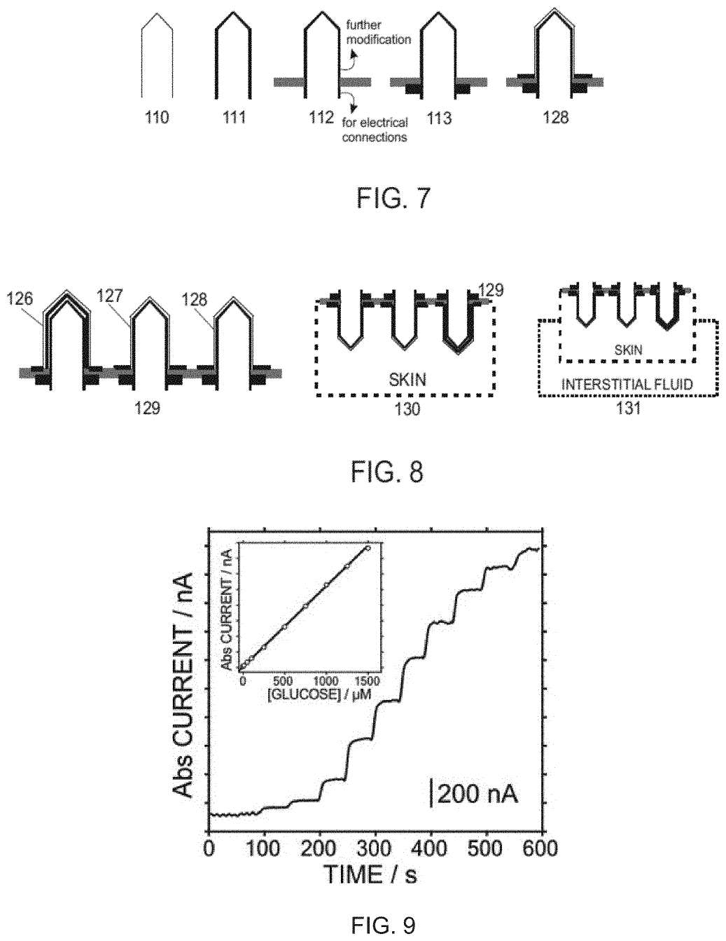

According to one embodiment, the method comprises providing at least one microneedle or needle (110) structured to function as a pseudocounter electrode (128) for molecule sensing, coating said at least one microneedle or needle with a coating to improve the conductivity of the microneedle or needle, and providing a constant long-term electrochemical potential, and thereby serving as a base material of the pseudocounter electrode. The at least one microneedle or needle is then attached to the substrate of said (micro)needle-based patch and coating the coating to improve the biocompatibility with an external polymeric film, e.g., a polyurethane (PU) film (see FIG. 7).

According to another embodiment, the method of coating the working electrode (115) for ion sensing comprises coating the coating to improve the conductivity with an ion-to-electron transducer layer (114) and coating the ion-to-electron transducer layer with an ion-selective membrane (115). As an alternative, the method of coating the working electrode (126) for molecule sensing comprises coating the coating to improve the conductivity with a mediator layer (124) and coating the mediator layer with an enzyme film (125), and coating the enzyme layer with an additional external film for different purposes (i.e., skin penetration, interference barrier, biocompatibility improvement, etc.) (126).

However, the first coating to improve the conductivity (111) can be suppressed and the microneedle or needle can be directly modified through a chemical procedure to implement the sensing element. In the case of ions detected with potentiometry, the preferred example (but being not restricted) considers that the sensing element is a redox-active self-assembled monolayer. Then, the ion-selective membrane can be coated on top of the redox-active self-assembled monolayer (126). In a variation of this approach from some to all the elements of the ion-selective membrane are also forming part of the self-assembled monolayer. For example, a mixed monolayer containing redox-active moieties together with an ion-selective ionophore.

The same definition applies to the case of molecule detection. The mediator layer (124) can be chemically attached to the microneedle or needle without the need of the said layer to improve conductivity. In this case, the mediator layer can be a redox active self-assembled monolayer. The monolayer can additionally contain the enzyme in a mix format together with the redox-active moieties. The monolayer is then coated with the said additional external film with different purposes (126).

In another embodiment, the method of coating the reference electrode (120) for ion sensing comprises coating the coating to improve the conductivity (116) with a reference membrane film (119), and coating the reference membrane film with an external polymeric film, e.g., a polyurethane (PU) film (120). As an alternative, the method of coating the pseudoreference electrode (127) for molecule sensing comprises coating the coating to improve the conductivity (116) with an external polymeric film, e.g. a polyurethane (PU) film (127), i.e. without a reference membrane.

For the potentiometric readout of an ion, a working electrode and a reference electrode configured as microneedles or needles in the type of 115 and 120, respectively, are implemented in the (micro)needle-based patch (120). The (micro)needle-based patch is fashioned for transdermal detection by skin insertion (122) and can be configured to contain different kind of microneedles and needles as well as various electrochemical readouts (2 and 4 respectively). For the amperometric readout of a molecule, a working electrode, a pseudocounter electrode and a pseudoreference electrode configured as microneedles or needles in the type of 126, 127 and 128, respectively, are implemented in the (micro)needle-based patch (129). The (micro)needle-based patch is fashioned for transdermal detection by skin insertion (130) and can be configured to contain different kind of microneedles and/or needles as well as various electrochemical readouts (2 and 4 respectively).

The purpose of the substrate in the (micro)needle-base patch is to provide a platform to fix the length of the microneedles or needles for effective transdermal insertion in further on-body portability. Different dimensions and forms can be selected depending on the final aim of the patch. For example, to reach the interstitial fluid, a length of less than 1000 μm and preferably through microneedles. To reach the blood stream, a length higher than 1500 μm is necessary and preferably through needles.

The polymeric nature of the substrate allows for an adequate on-body portability and the adequate sealing of all the coatings needed for the microneedle/needle modification. The preparation protocols for the sensing microneedles and needles through all the variations of method 1 are designed to retain the external modification before, during and after skin penetration. In any case, the first modification of the microneedle or needle with the purpose of increasing the conductivity (111, 116) or with the self-assembled monolayer are accomplished before the integration in the patch. Then, 111 or 116 are inserted and glued in the polymeric substrate (112+113 for the any working electrode, 117+118 for the reference and pseudoreference electrode, 112+113 for the pseudocounter electrode). Next, the rest of components providing the sensing purpose, if necessary, are incorporated (114 for the working electrode for ions, 119 for the reference electrode, 124 and 125 for the working electrode for molecules). Finally, an external polymeric layer preferably (but not restricted to) made of PU assures the fixation of all the external modification to the substrate (115 in the working electrode for ion detection, 120 in the reference electrode, 126 in the working electrode for molecule detection, 127 in the pseudoreference electrode, 128 in the pseudocounter electrode). This layer also facilitates skin penetration. This layer may also have other side purposes, such as interference repulsion or a diffusion barrier for the analyte in the working electrode for molecules.

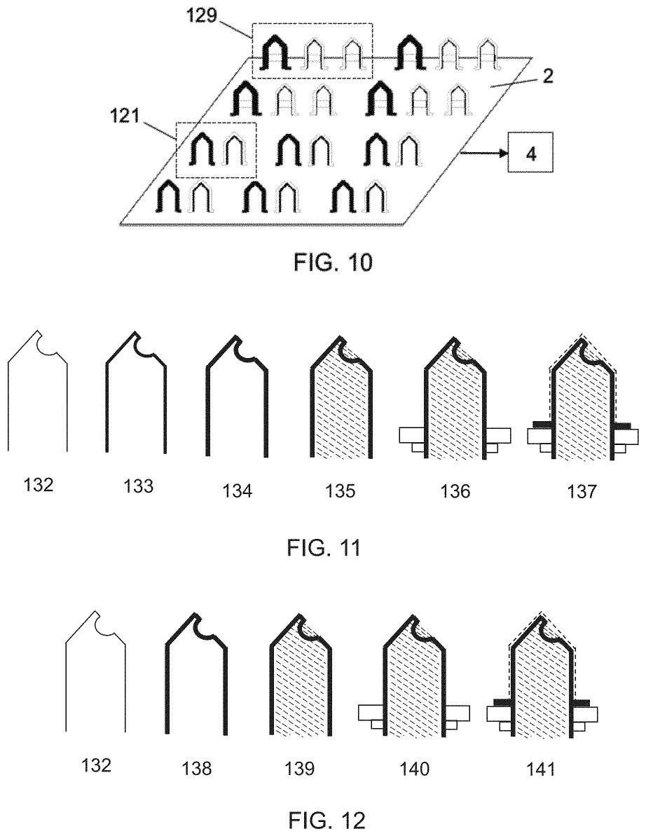

In FIG. 10 is schematically illustrated a (micro)needle-based patch 2 configured for ion and/or molecule on-body transdermal sensing, comprising at least two microneedles or needles modified according to any of the method steps of method 1 which have been described above, and which will be further described in the following. The microneedle patch 2 is configured to be applied for any electrochemical technique, e.g., potentiometry and amperometry, and for any analyte, and wherein the patch is configured to be connected to a device 4 adapted to process sensed electrochemical values.

Various considerations and aspects of method 1 will now be described.

Ion detection with potentiometry may be particularly performed by a working electrode and reference electrode prepared as described in the following.

The method of modifying a microneedle to a working electrode is illustrated in FIG. 1. A microneedle or needle (110), for example made from solid stainless-steel, is modified with carbon ink or any other material (e.g., metals, doped silicon, etc.) to increase the conductivity (111, this can be done by dip coating, spraying, drop-casting, electroplating, etc.). In particular, the carbon layer is cured in the oven (120° C., 10 min). The carbon-coated microneedle or needle is preferably embedded into the polymeric substrate (112), for example made of polydimethylsiloxane (PDMS), and the external part of the microneedle or needle (the one that is not going to be further modified) was glued to the substrate (113) to avoid detachment and finally left to dry at room temperature for 4 h.

The film of the ion-to-electron transducer is deposited on top of the carbon-modified microneedle or needle (114). The ion-to-electron transducer can be dissolved in a solution of an organic solvent easy and fast to be evaporated after drop-casting, dip coating of spraying deposition, as the case of nanomaterials, conducting polymers or redox buffers. The ion-to-electron transducer can be electropolymerized on the carbon-modified microneedle or needle, as the case of conducting polymers. The ion-to-electron transducer can be a redox-active self-assembled monolayer prepared on the carbon-modified microneedle or needle, but also directly on the microneedle or needle without the need of the carbon coating. For example, solid glassy carbon microneedles or needles can be modified with a ferrocene-based monolayer by electrodeposition of a diazonium salt and inserting ethynilferrocene by click chemistry reaction. However, any monolayer can be optimized in the form of microneedle and needle by adjusting the used materials and (electro)chemical reactions.

Finally, the ion-selective membrane cocktail can be drop-casted, sprayed or dip coated on top of the ion-to-electron transducer (115). The membrane cocktail generally comprises a polymer (e.g., PVC, PU, etc.), a plasticizer (e.g., DOS, NPOE, etc.), an ion-exchanger (e.g., NaTFPB for cations, TDMACl for anions, etc.) and the corresponding receptors (or ionophore) for the ion analyte dissolved in THF. This allows for the formation of a plasticized polymeric membrane after the evaporation of the THF. However, any kind of ion-selective membrane with any composition can be implemented here (e.g., plasticizer-free membranes, Nafion films, etc.). The (micro)needle-based ion-selective electrode was conditioned overnight in the analyte of interest at 1 mM concentration in water, or any other solution. Because this is the most external layer of the modified electrode and therefore will be in direct contact with the skin and tissues during on-body portability, all the materials utilized in this coating assure biocompatibility.

Depending on the length of the microneedle or needle, the working electrode will reach the interstitial fluid (less than 1000 μm) or blood (>1500 μm reaching the bloodstream). Owing to a reduced number of layers in the preparation of the (micro)needle-based working electrode, the original size of the diameter of a (micro)needle is only incremented as much as 100 μm in total after the entire external modification. This increase is much lower when the required elements for the sensing elements are chemically attached to the microneedle or needle (e.g., the self-assembled monolayer) rather than physically deposited. Consequently, implementation of the present invention assures compatibility with painless insertion. The addition of any extra layer needed for the appropriate ion sensing, interference suppressing, biocompatibility enhancement and/or penetration facilitation is possible while minimizing pain in the individual.

FIG. 1 discloses:
110: solid microneedle or needle.
111: microneedle or needle with carbon coating.
112: microneedle or needle with carbon coating implemented in the polymeric substrate.
113: microneedle or needle with carbon coating implemented and glued in the polymeric substrate.
114: addition of the ion-to-electron transducer film.
115: addition of the ion-selective membrane external film.

The method of modifying a microneedle or needle to a reference electrode, for example made from solid stainless-steel, is illustrated in FIG. 2.

The solid microneedle or needle (110) is modified with Ag/AgCl ink (116, this can be done by dip coating, spraying or drop-casting). The Ag/AgCl layer is cured in the oven (120° C., 10 min). This step allows having a pseudoreference electrode. The Ag/AgCl-coated (micro)needle is embedded into the polymeric substrate (117), for example made of polydimethylsiloxane (PDMS), and the external part of the microneedle or needle (the one that is not going to be further modified) was glued to the substrate (118) to avoid detachment and finally left to dry at room temperature for 4 h. A film of the reference membrane is then deposited on top of the carbon-modified microneedle or needle (119) by drop-casting, dip coating or spraying a cocktail of the components in a volatile organic solvent. Finally, a layer of a biocompatible polymer (preferably polyurethane but also Nafion, etc.) is incorporated by drop-casting, dip coating or spraying a solution of this material in a volatile organic solvent. This last step avoids the leaching of the salt (KCl) included in the reference membrane and improves the potential stability of the reference electrode. The reference electrode 120 fulfils all the requirements established for a reference electrode in terms of providing a constant potential for increasing concentrations of salts, redox species, pH changes, light changes and a minimum drift in the order of tens of microvolts per hour.

The reference electrode 120 is to be used together with the working electrode 115 in a (micro)needle-based patch for transdermal detection of ions with potentiometric readout. In other variants, the reference electrode can be used together with any other working electrode. Depending on the length of the microneedle or needle, the reference electrode will reach the interstitial fluid (less than 1000 μm) or blood (>1500 μm reaching the bloodstream). Owing to a reduced number of layers in the preparation of the sensors the original size of the (micro)needle is only incremented as much as 100 μm in total. Consequently, the proposed fabrication assures compatibility with painless insertion. Thus, the addition of any extra layer needed for the appropriate functioning of the reference electrode, interference suppressing, biocompatibility enhancement and/or penetration facilitation is possible while minimizing pain in the individual.

FIG. 2 discloses:
110: solid microneedle or needle.
116: microneedle or needle with Ag/AgCl coating.
117: microneedle or needle with Ag/AgCl coating implemented in the polymeric substrate.
118: microneedle or needle with Ag/AgCl coating implemented and glued in the polymeric substrate.
119: addition of the reference membrane film.
120: addition of the PU film.

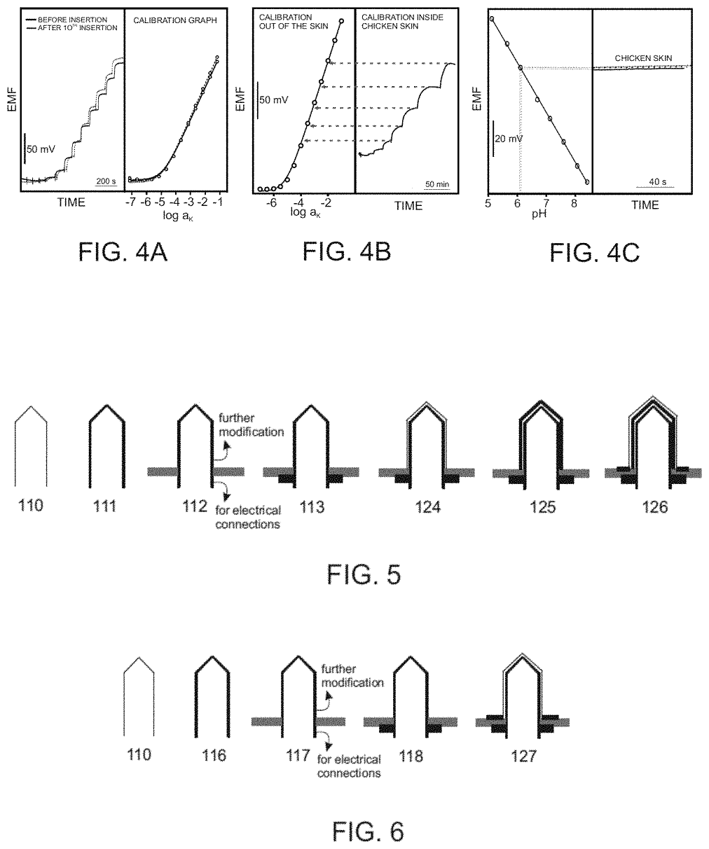

In FIG. 3 is illustrated a (micro)needle-based patch for transdermal ion detection. In one specific example, we demonstrate some analytical performances for a patch comprising potassium-selective microneedle (115) together with the reference electrode (120). For this purpose, (115) comprises carbon nanotubes as the ion-to-electron transducer (114) and a potassium-selective membrane with the potassium ionophore valinomycin (working electrode). For the preparation of (115), functionalized multiwalled carbon nanotubes (f-MWCNTs) were deposited on top of (113) by drop casting 10×4 μL of a f-MWCNTs solution in THF (1 mg mL⁻¹), allowing each layer to be dried for 2 min before depositing the next one. For the ion-selective membrane, a THF cocktail based on PU, DOS, KTFPB and valinomycin was used: 4 μL of the membrane cocktail on top of the ion-to-electron transducer film and left to dry at room temperature for 4 h until the total evaporation of the THF. For the preparation of (120), the reference membrane cocktail is a polymeric one comprising poly(vinyl butyral) (PVB) and is applied as 3×4 μL, and allowing each layer to dry for 10 min. The reference membrane is left to dry overnight. The reference electrode was conditioned for 12 h in 3 M KCl and left to dry in air for 1 h. Finally, the PU layer is added: 4 μL of polyurethane solution (PU, 30% in THF) is drop cast onto the microneedle and left to dry at room temperature for 4 h. Both (115) and (120) are simultaneously incorporated in the PDMS substrate (121). The electrode (115) was conditioned in 1 mM KCl solution. The calibration graph of the microneedle patch is performed for increasing concentrations of potassium in artificial interstitial fluid, as shown in FIG. 4A. The electrodes displayed the expected Nernstian behavior that is well-established for potentiometric sensors. In this specific example, the calibration graph is repeated after 10 insertions in chicken skin (122) to confirm the adequate robustness of the sensors manufacturing. After 10 insertions the calibration parameters remained constant. The linear range of response is suitable for the transdermal detection of potassium in interstitial fluid according to the expected levels in humans. The same experiment can be accomplished in artificial serum/blood to evaluate the analytical performances of longer microneedles for transdermal blood analysis. The same experiment may be accomplished for other ion-selective membranes for the transdermal detection of other ions in interstitial fluid and/or blood.

FIG. 3 discloses:
121: patch comprising modified microneedles or needles as working and reference electrode (115 and 120, respectively) for transdermal ion detection by means of potentiometric readout.
122: patch penetrating the skin.
123: microneedle patch penetrating the skin and in contact with artificial interstitial fluid in which increasing concentration of the ion analyte are added. The same scheme is translated into a needle patch penetrating the skin and in contact with artificial blood.

The potassium-selective microneedle is suitable for the monitoring of fluctuations of potassium levels in interstitial fluid while the patch is penetrating the skin (123). FIG. 4B shows one calibration graph accomplished before penetration of chicken skin and the dynamic potential recorded with the microneedle patch penetrating the skin while varying potassium ion concentration in the interstitial fluid. A comparison of the recorded potential with the calibration graph provides the transdermal potassium concentration. The same experiment can be accomplished for other ion-selective membranes for the transdermal detection of other ions in interstitial fluid. For example, FIG. 4C displays a calibration graph for a microneedle pH sensor and three replicate transdermal measurements accomplished in chicken skin overnight conditioned in a buffer pH solution of pH=6.1. Analytical performances are evaluated to ensure the detection of the ion analyte concentration in interstitial fluid (or blood). The tailoring of the working electrode (115) may be modified in order to reach the desired analytical performances.

The (micro)needle patch for transdermal ion detection may incorporate as many working electrodes (115) as desired. The limiting factor here is that the patch becomes bigger with more working electrodes. The user decides the compromise between the final size of the patch and the number of working electrodes (115). In one specific example, the patch has a common reference electrode (120) for all the working electrodes (115). There are three possibilities here: (i) all the electrodes 115 are prepared with the same ion-selective membrane in order to obtain the reproducibility of the transdermal measurements; (ii) every electrode 115 is fabricated with a different ion-selective membrane according to the ion analyte, here it is possible to measure cations and anions simultaneously; and the last option (iii) is a combination if (i) and (ii). In one variation of this example, the modified microneedles and needles are of different lengths to reach the interstitial fluid and the bloodstream simultaneously. This patch has applicability for the measurements of the same analyte in both biological fluids aiming at finding correlations with clinical relevance. The microneedle patch 121 for transdermal detection of ions is compatible with any electrical module. It is here preferred the implementation of miniaturized PCB circuit boards for the wireless transmission of the measurements to a device for data harvesting, treatment and interpretation during on-body portability.

FIG. 4 discloses the response at increasing concentrations (calibration graph) for microneedles structured for the potentiometric detection of potassium ions in artificial interstitial fluid before and after skin penetration using a microneedle patch (FIG. 4A); the response at increasing concentrations for potassium ions before and during skin penetration (FIG. 4B); and the response of chicken skin pieces conditioned at a fixed pH together with the calibration graph of the pH microneedle sensor (FIG. 4C).

FIGS. 5-8 illustrates modification of microneedles or needles to be applied for molecules detection with amperometry by a working electrode, a pseudoreference electrode and a pseudocounter electrode.

The method of modifying a microneedle or needle into a working electrode for molecule sensing is illustrated in FIG. 5.

A solid microneedle or needle (110), for example made of (but not restricted to) stainless-steel, is modified with carbon ink or other conductive material (111), which can be done by dip coating, spraying, drop-casting or electroplating. The carbon layer is cured in the oven (120° C., 10 min). The carbon-coated microneedle or needle is embedded into the polymeric substrate (112), for example made of polydimethylsiloxane (PDMS), and the external part of the microneedle or needle (the one that is not going to be further modified) was glued to the substrate (113) to avoid detachment and finally left to dry at room temperature for 4 h. Then, a film of a mediator needed for the amperometric measurement in enzyme-based sensors is deposited (e.g., by electropolymerization, drop casting, etc. depending on the nature of the mediator) on top of the carbon-modified microneedle or needle (124). Subsequently, the enzyme film is added (e.g., by drop casting) (125). Finally, an external layer is added (126). The function of the external layer varies according to the molecule to be analyzed. The external layer can be of different materials (chitosan, polyurethane, Nafion, etc.) and for different purposes (enzyme entrapment, biocompatibility, interference barrier, etc.). All these materials are prone to provide appropriate sealing of all the coatings through a good interaction with the polymeric substrate as well as biocompatibility. Depending on the nature of the microneedle/needle material, the first film of a conductive material is not needed and the redox mediator can be directly included in the microneedle/needle architecture. For example, for a glassy carbon microneedle or needle the mediator Prussian blue can be directly deposited on top. Also, for a glassy carbon microneedle, a self-assembled monolayer obtained through click chemistry can act as the redox mediator in the enzyme-base working electrode.

Depending on the length of the microneedle or needle, the working electrode will reach the interstitial fluid (less than 1000 μm) or blood (>1500 μm reaching the bloodstream). Owing to a reduced number of layers in the preparation of the (micro)needle-based working electrode, the original diameter of the microneedle or needle is only incremented as much as 100 μm in total. Consequently, the proposed fabrication assures compatibility with painless insertion. Thus, the addition of any extra layer needed for the appropriate ion sensing, interference suppressing, biocompatibility enhancement and/or penetration facilitation is possible while minimizing pain in the individual.

FIG. 5 discloses:
110: solid microneedle or needle.
111: microneedle or needle with carbon coating.
112: microneedle or needle with carbon coating implemented in the polymeric substrate.
113: microneedle or needle with carbon coating implemented and glued in the polymeric substrate.
124: addition of the mediator film.
125: addition of the enzyme layer.
126: addition of the external layer.

The method of modifying a microneedle into a pseudoreference electrode is illustrated in FIG. 6.

A solid microneedle or needle (110), for example made of (but not restricted to) stainless-steel, is modified with Ag/AgCl ink (116, this can be done by dip coating, spraying or drop-casting). The Ag/AgCl layer is cured in the oven (120° C., 10 min). This step allows having a pseudoreference electrode. The Ag/AgCl-coated microneedle is embedded into the polymeric substrate (117), for example made of polydimethylsiloxane (PDMS), and the external part of the microneedle or needle (the one that is not going to be further modified) was glued to the substrate (118) to avoid detachment and finally left to dry at room temperature for 4 h. Then, a film of polyurethane (or other biocompatible polymer) is deposited on top of the carbon-modified microneedle (127) by drop-casting, dip-coating or spraying a solution in a volatile organic solvent. This last step avoids the leaching of the salt (KCl) and improves the potential stability of the reference electrode. The pseudoreference electrode (127) fulfils all the requirements established for a pseudoreference electrode in terms of providing a constant potential for increasing concentrations of salts, redox species, pH changes, light changes and a minimum drift in the order of tens of microvolts per hour. The pseudoreference electrode (127) is to be used together with the working electrode (126) in a microneedle patch for transdermal detection of (bio)molecules.

Depending on the length of the microneedle or needle, the pseudoreference electrode will reach the interstitial fluid (less than 1000 μm) or blood (>1500 μm reaching the bloodstream). Owing to a reduced number of layers in the preparation of the (micro)needle-based pseudoreference electrode, the original size diameter of the microneedle or needle is only incremented as much as 100 μm in total. Consequently, the proposed fabrication assures compatibility with painless insertion. Thus, the addition of any extra layer needed for the appropriate functioning of the pseudoreference electrode, interference supressing, biocompatibility enhancement and/or penetration facilitation is possible while minimizing pain in the individual.

FIG. 6 discloses:
110: solid microneedle or needle.
116: microneedle or needle with Ag/AgCl coating.
117: microneedle or needle with Ag/AgCl coating implemented in the polymeric substrate.
118: microneedle or needle with Ag/AgCl coating implemented and glued in the polymeric substrate.
127: addition of the PU film.

The method of modifying a microneedle or needle into a pseudocounter electrode is illustrated in FIG. 7.

A solid microneedle or needle (110), for example made of (but not restricted to) stainless-steel, is modified with carbon ink (111), which can be done by dip coating, spraying or drop-casting. The carbon layer is cured in the oven (120° C., 10 min). The carbon-coated (micro)needle is embedded into the polymeric substrate (112), for example made of polydimethylsiloxane (PDMS), and the external part of the (micro)needle (the one that is not going to be further modified) was glued to the substrate (113) to avoid detachment and finally left to dry at room temperature for 4 h. Finally, an external coating of a biocompatible polymeric material (i.e., PU, Nafion and others) is added by drop-casting, dip coating or spraying a solution in a volatile organic solvent (128). The pseudocounter electrode (128) fulfils all the requirements established for a pseudocounter electrode in terms of being inert. The pseudocounter electrode (128) is to be used together with the working electrode (126) and the pseudoreference electrode (127) in a (micro)needle patch for transdermal detection of (bio)molecules.

Depending on the length of the microneedle, the pseudocounter electrode will reach the interstitial fluid (less than 1000 μm) or blood (>1500 μm reaching the bloodstream). Owing to a reduced number of layers in the preparation of the (micro)needle-based pseudocounter electrode, the original size of the diameter of the microneedle or needle is only incremented as much as 100 μm in total. Consequently, the proposed fabrication assures compatibility with painless insertion. Thus, the addition of any extra layer needed for the appropriate functioning of the pseudoreference electrode, interference suppressing, biocompatibility enhancement and/or penetration facilitation is possible while minimizing pain in the individual.

FIG. 7 discloses:
110: solid microneedle or needle.
111: microneedle or needle with carbon coating.
112: microneedle or needle with carbon coating implemented in the polymeric substrate.
113: microneedle or needle with carbon coating implemented and glued in the polymeric substrate.
128: addition of the external polymeric layer.

The (micro)needle-based patch for molecule detection is illustrated in FIG. 8. In one specific example, we demonstrate some analytical performances for a patch comprising glucose microneedle sensor (126) together with the pseudoreference electrode (127) and pseudocounter electrode (128). The three electrodes are simultaneously incorporated in the polymeric substrate (129). The glucose sensor (126) comprises a layer of iron hexacyanoferrate (Prussian blue) as the mediator (124), glucose oxidase as the enzyme layer (125) and Nafion as the external layer (126). The specific procedure for the mediator deposition (124) was as follows: 1 μL of 0.1M potassium ferricyanide in 0.01M HCl+2 μL of 0.1M FeCl₃ in 0.01M HCl, mixed both solutions, drop-casting on the carbon-coated microneedle and let dry for 20 min at room temperature. Rinse with 10 μL of 1 mMHCl. Anneal the modified microneedle for 1 h at 100° C. The specific procedure for the enzyme immobilization (125) was as follows: solution of 1% chitosan in 0.1M acetic acid, solution of glucose oxidase (30 mg/mL+10 mg/mL bovine serum albumin in phosphate buffer 10 mM at physiological pH), mix both solutions at 2:1 v:v, drop-cast 1 μL of this solution on top of the mediator and let it dry for 30 min in the oven. For the external layer (126), 0.5 μL of Nafion dispersion in water (1 wt. %) and let it dry for 20 min at room temperature in the fume hood. It is convenient to storage this electrode in the fridge at 4° C. when not in use. For the preparation of the pseudoreference electrode (127) and the pseudocounter electrode (128), the external layer was PU prepared as in (126).

FIG. 8 discloses:
129: patch comprising modified microneedles or needles as working, pseudoreference and pseudocounter electrode (126, 128, and 127, respectively) for transdermal molecule detection by means of amperometric readout.
130: patch penetrating the skin.
131: patch penetrating the skin and in contact with artificial interstitial fluid in which increasing concentrations of the analyte are added. The same scheme is translated into a needle patch penetrating the skin and in contact with artificial blood.

The calibration graph of the microneedle patch that is performed for increasing concentrations of glucose at physiological pH medium, is shown in FIG. 9. The linear range of response is compatible with the expected levels of glucose in both interstitial fluid and blood. Although not shown, whether this calibration graph is repeated after several penetrations in animal skin the calibration parameters are to be maintained. The same experiment concerning the calibration graph can be accomplished with other microneedles or needles accordingly modified for the transdermal detection of other molecules (such as lactate, hormones, creatinine, etc.). The glucose microneedle is suitable for the monitoring of fluctuations of glucose levels in interstitial fluid while the patch is penetrating the skin (130). A calibration graph performed before penetration in the skin allows for the conversion of the dynamic potential recorded with the (micro)needle patch penetrating the skin while varying glucose concentration in the interstitial fluid. As a result, a comparison of the recorded potential with the calibration graph provides the transdermal glucose concentration, in total analogy with the potassium-selective microneedle patch (121) (see FIG. 9). Analytical performance of every individual molecule microneedle or needle is to be evaluated to ensure the transdermal detection of the analyte concentration in interstitial fluid (or blood). The working electrode (126) may be modified in order to reach the desired analytical performances.

The (micro)needle patch for transdermal molecule detection may incorporate as many working/pseudoreference/pseudocounter electrodes (129) as desired. Multi-molecule transdermal detection in amperometric mode is different than the multi-ion transdermal detection in potentiometric mode. For multi-molecule detection, every working electrode has its own pseudoreference and pseudocounter electrodes to avoid cross-talking and other kind of chemical interferences. The limiting factor here is that the patch becomes bigger with more bunches of electrodes (129). The user decides the compromise between the final size of the patch and the number of analytes to be detected. There are three possibilities here: (i) all the working electrodes (126) are prepared for the same analyte in order to obtain the reproducibility of the transdermal measurements; (ii) every working electrode (126) is fabricated for a different analyte; and the last option (iii) is a combination if (i) and (ii).

In another variation, the (micro)needle patch may combine both ion and molecule detection with potentiometry and amperometry mode respectively. In one specific example, the modified microneedles and needles are of different lengths to reach the interstitial fluid and the bloodstream simultaneously by means of the same patch. One of the applications of this patch is for the measurements of the same analyte in both biological fluids aiming at finding correlations with clinical relevance.

The (micro)needle patches for transdermal detection of molecules and molecules/ions are compatible with any electrical connections. It is here of interest the implementation of miniaturized PCB circuit boards for the wireless transmission of the measurements to a device for data harvesting, treatment and interpretation.

FIG. 9 discloses a calibration graph for glucose obtained by means of a glucose microneedle sensor.

FIG. 10 discloses a patch 2 for multi-analyte detection mixing potentiometric, and amperometric, (micro)needle-based sensors for transdermal analysis of ion and/or molecules in interstitial fluid and/or blood. In the figure four sets of (micro)needles for molecule detection (129), and six sets of (micro)needles for ion detection (121), is illustrated. In the figure is also illustrated the connection with a device 4 for analysis of the obtained electrochemical sensor signals.

### Method 2— Internal Modification of Hollow Microneedles and Needles

The present invention relates to a method, method 2, of modifying the surfaces of inner walls inside at least one hollow microneedle or needle, or the surfaces of at least one conductive wire to be introduced inside a hollow microneedle or needle. Each microneedle/needle may act as an individual electrode or each microneedle/needle may contain all the electrodes needed for the electrochemical readout: the working electrode and reference electrode for amperometry; the working electrode, pseudocounter electrode and pseudoreference electrode for amperometry. The microneedle or needle is internally compartmented in such way that the inner modification related to method 2, either by modifying the wall or with the introduction of a modified wire, is referred to at least two compartments inside a hollow microneedle or needle. The microneedle or needle compartmentation is essential to avoid the presence of mixed potentials, cross-talking and other interferences. The compartments will be generated by insulator materials if necessary, to avoid this sort of interferences. The at least one microneedle or needle internally modified is to be arranged in a (micro)needle-based patch configured for ion and/or molecule on-body transdermal sensing. Method 2 will be described with references to FIGS. 11-18.

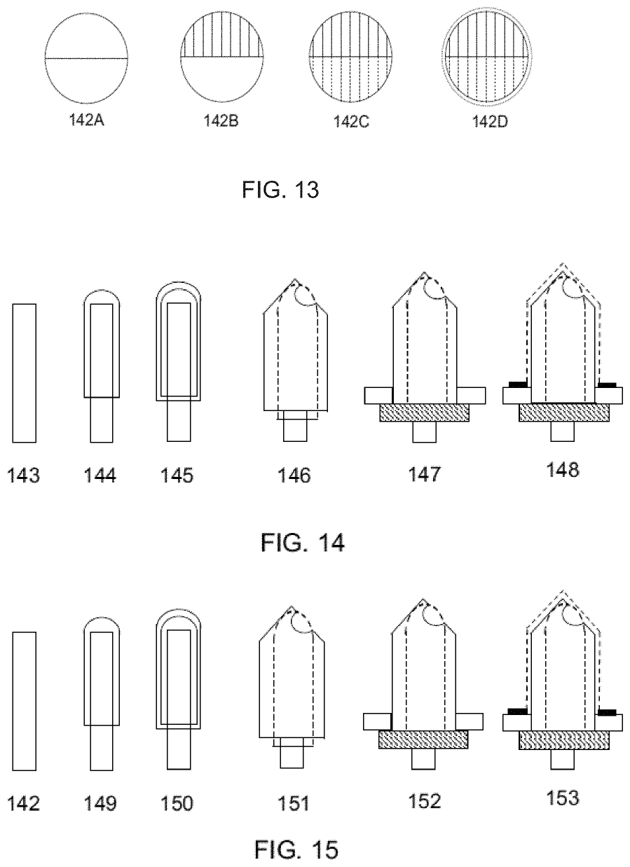

The method comprises providing an inner surface of a (compartmented) hollow microneedle or needle (132 or 142A), or a surface of a conductive wire (143) to be introduced inside a (compartmented) hollow microneedle or needle, the surface is structured to function as a working electrode (137 and 142C, 147 and 154C) for ion sensing or molecule sensing, coating said surface with a coating to improve the conductivity of the microneedle or needle, and attaching the microneedle or needle to a substrate of said (micro)needle-based patch (see FIGS. 11 and 13). The method further comprises providing a surface of a (compartmented) hollow microneedle or needle, or a surface of a conductive wire to be introduced inside a (compartmented) hollow microneedle or needle, the surface is structured to function as a reference electrode (141 and 142C, 153 and 154C) for ion sensing, or as a pseudoreference electrode for molecule sensing, and coating the surface with an Ag/AgCl layer coating to improve the conductivity of the microneedle or needle and thereby serving as a reference electrode or pseudoreference electrode, and attaching the (micro)needle to the substrate of said microneedle-based patch (see FIGS. 12 and 15).

In a further embodiment the method comprises providing an inner surface of a compartment of a hollow microneedle or needle, or a surface of a conductive wire to be introduced inside a (compartmented) hollow microneedle needle, the surface is structured to function as a pseudocounter electrode for molecule sensing, coating said surface with a coating to improve the conductivity of the microneedle or needle and providing a constant long-term electrochemical potential and thereby serving as a base material of the pseudocounter electrode, and attaching the at least one microneedle or needle to the substrate of said (micro)needle-based patch, and coating the coating to improve the conductivity with an external polymeric film, e.g. a polyurethane (PU) film.

According to another embodiment, the method of coating the surface to function as working electrode (137, 148) for ion sensing comprises coating the coating to improve the conductivity (133) with an ion-to-electron transducer layer (134) and coating the ion-to-electron transducer layer with an ion-selective membrane (135). After being fixed in the polymeric substrate (136), an external layer is added to seal the microneedle or needle with the substrate (137). The method of coating the surface to function as working electrode for molecule sensing comprises coating the coating to improve the conductivity (133) with a mediator layer (134), and coating the mediator layer with an enzyme film (135), and coating the enzyme layer with an additional external film (137) for different purposes in addition to the sealing of the microneedle or needle with the substrate (i.e., skin penetration, interference barrier, biocompatibility improvement, etc.).

In another embodiment, the method of coating the surface to function as reference electrode (141, 153) for ion sensing comprises coating the coating to improve the conductivity (138) with a reference membrane film (139) and coating the reference membrane film with an external polymeric film (141), e.g., a polyurethane (PU) film, after being implemented in the substrate. The method of coating the surface to function as pseudoreference electrode for molecule sensing comprises coating the coating to improve the conductivity with an external polymeric film, e.g., a polyurethane (PU) film.

Analogous to method 1, depending on the material of the microneedle or needle, the first coating to improve the conductivity is not necessary and the rest of the layers can be developed directly on the microneedle or needle. For example, this is the case of glassy carbon microneedles and needles. All the considerations in this regard already described for method 1, are applicable to method 2 in the fabrication of any type of electrode related to this invention.

In all the cases, the internal modification can be accomplished until the end of the (micro)needle tip or at some specific length far from the tip. In the first option, the sensing element will be in direct touch with the biological fluid. In the second option, the biological fluid is expected to reach the sensing element in the deep interior of the (micro)needle, therefore filling in an internal cavity destined to the sample. Both options fit with all the electrode description provided for method 2.

The schematic FIG. 18 illustrates a (micro)needle-based patch 6 configured for ion and/or molecule on-body transdermal sensing that comprises at least one microneedle or needle 10 modified according to method 2 as described above and further described in the following. The (micro)needle patch is configured to be applied for any electrochemical technique, e.g., potentiometry and amperometry, and for any analyte, and wherein the patch is configured in such a way to be connected to a device 8 adapted to process sensed electrochemical values. Depending on the length of the microneedle or needle, the working electrode will reach the interstitial fluid (less than 1000 μm) or blood (>1500 μm reaching the bloodstream).

Various considerations and examples of method 2 will now be described.

Ion detection with potentiometry may be performed by a working electrode and reference electrode.

Method 2 relates to the modification of the internal walls of a hollow microneedle or needle, which will be illustrated in FIGS. 11 and 12.

FIG. 11 illustrates modification of a hollow microneedle or needle to a working electrode.

A hollow microneedle or needle (132), for example made of (but not restricted to) stainless-steel, is internally modified with carbon ink (133, this can be done by spraying or drop-casting). The carbon layer is cured in the oven (120° C., 10 min). The carbon-modified microneedle or needle is internally modified with a dispersion of the ion-to-electron transducer (134), which can be done by spraying or drop-casting. The ion-to-electron transducer is dissolved in a solution of an organic solvent easy and fast to be evaporated. The modified microneedle or needle is in turn modified with the ion-selective membrane cocktail by drop-casting the corresponding cocktail through the hole (135), i.e., the needle tip. This procedure is to be accomplished with the microneedle or needle placed in horizontal position in order to allow the needle to be filled with the cocktail. The membrane cocktail generally comprises a polymer (PVC, PU), a plasticizer (DOS, NPOE), an ion-exchanger (NaTFPB for cations, TDMACl for anions) and the corresponding receptors of ionophore for the ion analyte dissolved in THF. This allows for the formation of a plasticized polymeric membrane after the evaporation of the THF. However, any kind of ion-selective membrane with any composition can be here implemented. The modified microneedle or needle is then embedded into the polymeric substrate, for example made of polydimethylsiloxane (PDMS), and the external part of the microneedle or needle is glued to the substrate (136) to avoid detachment and left to dry at room temperature for 4 h. Finally, an external coating is added to seal the modified microneedle or needle. The film is conveniently based on PU material (137). In a variation of this procedure, and only for some specific ions, the ion-to-electron transducer and the membrane can be added in the same cocktail. In another variation of this procedure and, depending on the material of the microneedle or needle, the ion-to-electron transducer is chemically attached directly to the microneedle or needle, without any initial layer to improve the conductivity. Other components needed for the sensing element can also be chemically immobilized together with the ion-to-electron transducer, as detailed for method 1. The (micro)needle-based ion-selective electrode is finally conditioned overnight in the analyte of interest at 1 mM concentration in water or any other solution.

FIG. 11 discloses:
132: hollow microneedle or needle.
133: microneedle or needle with internal carbon coating.
134: microneedle or needle with carbon coating and the ion-to-electron transducer.
135: filling in with the ion-selective membrane.
136: implementation in the polymeric substrate.
137: addition of the external polymeric layer.

FIG. 12 illustrates modification of a hollow microneedle or needle to a reference electrode.

A hollow microneedle or needle (132), for example made of (but not restricted to) stainless-steel, is internally modified with Ag/AgCl ink (138), which may be done by spraying or drop-casting. The Ag/AgCl layer is cured in the oven (120° C., 10 min). This step allows having a pseudoreference electrode. The Ag/AgCl-modified microneedle or needle is internally modified with the reference membrane (139), which can be done by spraying or drop-casting. A cocktail of the components in a volatile organic solvent is used. This procedure is to be accomplished with the microneedle or needle placed in horizontal position in order to allow the whole of the microneedle to be filled with the cocktail. The modified microneedle or needle is embedded into the polymeric substrate, for example made of polydimethylsiloxane (PDMS), and the external part of the microneedle was glued to the substrate (140) to avoid detachment and finally left to dry at room temperature for 4 h. Finally, an external coating is added to seal the modified microneedle or needle. The film is conveniently based on PU material (141). The reference electrode (141) fulfils all the requirements established for a reference electrode in terms of providing a constant potential for increasing concentrations of salts, redox species, pH changes, light changes and a minimum drift in the order of tens of microvolts per hour. The reference electrode (141) is to be used together with the working electrode (137) in a (micro)needle patch for transdermal detection of ions.

Depending on the length of the microneedle or needle, the reference electrode will reach the interstitial fluid (less than 1000 μm) or blood (>1500 μm reaching the bloodstream).

In one variation, the hollow microneedle or needle is internally compartmented in two spaces (142A). The wall in one space is structured to fabricate the working electrode as per the described steps (142B). The wall in the other space is structured to fabricate the reference electrode as per the described steps (142C). The compartmented hollow microneedle or needle internally modified with the working electrode and the reference electrode is covered by an external polymeric layer, e.g., polyurethane (PU), to ensure transdermal insertion and biocompatibility (142D). At least one microneedle or needle of 142D type is to be implemented in the wearable (micro)needle patch for ion sensing.

FIG. 12 discloses:
132: hollow microneedle or needle.
138: microneedle or needle with internal Ag/AgCl coating.
139: fill in with the reference membrane.
140: implementation in the polymeric substrate.
141: addition of the external polymeric layer (green).

FIG. 13 discloses:
142A: cross-sectional view of compartmented hollow microneedle or needle.
142B: cross-sectional view of compartmented hollow microneedle or needle modified with the working electrode.
142C: cross-sectional view of compartmented hollow microneedle or needle modified with the working electrode and the reference electrode.
142D: cross-sectional view of compartmented hollow microneedle or needle modified with the working electrode and the reference electrode, view from the top of the hole after the deposition of the external layer (e.g., PU).

FIGS. 14 and 15 illustrate a method of modifying a sensing wire to be inserted into a hollow microneedle or needle.

FIG. 14 schematically illustrates modification of a wire into a working electrode. A conductive wire with an external diameter of approximately half of the internal diameter of the hollow microneedle or needle (143) is modified with the following layers. First, a layer of the ion-to-electron transducer (144) deposited by spraying, drop-casting or dip coating a solution of the ion-to-electron transducer in a volatile organic solvent. Alternatively, the ion-to-electron transducer can be deposited by electropolymerization, such as conducting polymers, or by any other electrochemical procedure. But also, self-assembled monolayers can be architecture in the wire. A layer of the ion-selective membrane is then added to the modified wire by spraying, drop-casting or dip-coating (145). The membrane cocktail generally comprises a polymer (PVC, PU), a plasticizer (DOS, NPOE), an ion-exchanger (NaTFPB for cations, TDMACl for anions) and the corresponding receptors of ionophore for the ion analyte dissolved in THF. This allows for the formation of a plasticized polymeric membrane after the evaporation of the THF. However, any kind of ion-selective membrane with any composition can be here implemented. The modified wire is embedded into the microneedle or needle. Importantly, one part of the modified wire perfectly fits in the whole of the hollow microneedle (146) and the penetration length can be adjusted until the tip or to generate a sample space inside the (micro)needle, as described above. The modified microneedle or needle is implemented in the polymeric substrate, for example made of polydimethylsiloxane (PDMS), and the external part of the microneedle or needle was glued to the substrate (147) to avoid detachment and finally left to dry at room temperature for 4 h. Finally, an external coating is added to seal the modified microneedle or needle. The film is conveniently based on PU material (148). In a variation of this procedure, and only for some specific ions, the ion-to-electron transducer and the membrane can be added in the same cocktail. The (micro)needle-based ion-selective electrode was conditioned overnight in the analyte of interest at 1 mM concentration in water or any other solution. The unmodified part of the wire is kept outside the microneedle or needle and it serves for further electrical connections. When the glue in (147) is applied, this will also seal the external part of the wire and therefore, this is totally fixed to the patch. Depending on the length of the microneedle or needle, the working electrode will reach the interstitial fluid (less than 1000 μm) or blood (>1500 μm reaching the bloodstream).

FIG. 14 discloses:
143: conductive wire.
144: wire with the ion-to-electron transducer.
145: coating of the ion-selective membrane.
146: implementation into the hollow microneedle or needle.
147: implementation in the polymeric substrate.
148: addition of the external polymeric layer.

FIG. 15 schematically illustrates modification of wire into a reference electrode. A conductive wire with an external diameter of approximately half of the internal diameter of the hollow microneedle or needle (143) is modified first with the Ag/AgCl coating (149) and then with the reference membrane (150). These layers can be formed by spraying, drop-casting or dip coating. The Ag/AgCl is cured in the oven (120° C., 10 min) before the deposition of the reference membrane. The modified wire is embedded into the microneedle or needle. Importantly, one part of the modified wire perfectly fits in the whole of the hollow microneedle (151) and the penetration length can be adjusted until the tip or to generate a sample space inside the (micro)needle, as described above. The modified microneedle or needle is implemented in the polymeric substrate, for example made of polydimethylsiloxane (PDMS), and the external part of the microneedle was glued to the substrate (152) to avoid detachment and finally left to dry at room temperature for 4 h. Finally, an external coating is added to seal the modified microneedle. The film is conveniently based on PU material (153). The unmodified part of the wire is kept outside the microneedle or needle and it serves for further electrical connections. When the glue in (152) is applied, it will also seal the external part of the wire and therefore, it is totally fixed to the patch. The reference electrode (153) fulfils all the requirements established for a reference electrode in terms of providing a constant potential for increasing concentrations of salts, redox species, pH changes, light changes and a minimum drift in the order of tens of microvolts per hour. The reference electrode (153) is to be used together with the working electrode (148) in a (micro)needle patch for transdermal detection of ions.

Depending on the length of the microneedle or needle, the reference electrode will reach the interstitial fluid (less than 1000 μm) or blood (>1500 μm reaching the bloodstream).

In one variation, the hollow microneedle or needle is compartmented in two spaces (142A). The wire to be inserted in one space is structured to fabricate the working electrode as per the described steps (142B). The wire to be inserted in the other space is structured to fabricate the reference electrode as per the described steps (142C). The dimensions of the utilized wires are selected to ensure its implementation in the compartments of the microneedle or needle after modification as described above. The compartmented hollow microneedle or needle internally modified with the working electrode and the reference electrode is covered by an external polymeric layer, e.g., polyurethane (PU), to ensure transdermal insertion and biocompatibility (142D). At least one microneedle 142D is to be implemented in the wearable (micro)needle patch for ion sensing.

FIG. 15 discloses:
143: conductive wire.
149: wire with the Ag/AgCl film.
150: coating of the reference membrane.
151: implementation into the hollow microneedle or needle.
152: implementation in the polymeric substrate.
153: addition of the external polymeric layer.

The (micro)needle patch, comprising modified hollow microneedles and/or needles, may be applied for ion detection.

In one specific example, we demonstrate some analytical performances for a patch comprising potassium-selective microneedle (137) together with the reference electrode (141), as well as, comprising potassium-selective microneedle (148) together with the reference electrode (153). For this purpose, 137 and 148 comprise carbon nanotubes as the ion-to-electron transducer and a potassium-selective membrane with the potassium ionophore valinomycin. For the preparation of (137) and (148), functionalized multi-walled carbon nanotubes (f-MWCNTs) were utilized by drop casting 1 μL×2 of a f-MWCNTs solution in THF (1 mg mL⁻¹), allowing each layer to be dried for 2 min before depositing the next one. For the ion-selective membrane, a THF cocktail based on PU, DOS, KTFPB and valinomycin was used: 5 μL of the membrane cocktail on top of the ion-to-electron transducer film and left to dry at room temperature for 4 h until the total evaporation of the THF. For the preparation of (141) and (153), the reference membrane cocktail is a polymeric one comprising poly(vinyl butyral) (PVB) and is applied as 2×5 μL, and allowing each layer to dry for 10 min. The reference membrane is left to dry overnight. The reference electrode was conditioned for 12 h in 3 M KCl and left to dry in air for 1 h. Finally, the PU layer is added to the patch with the modified microneedle: 4 μL of polyurethane solution is drop cast onto the microneedle and left to dry at room temperature for 4 h. Electrodes (137) and (141) are simultaneously incorporated in the PDMS substrate. Electrodes (148) and (153) are simultaneously incorporated in another PDMS substrate. The working electrodes (137) and (148) were conditioned in 1 mM KCl solution. The calibration graph of the two microneedle patches (137+141 and 148+153) is performed for increasing concentrations of potassium in artificial interstitial fluid, as shown in FIG. 17. The potassium microneedle sensors displayed a linear calibration graph comprising the expected levels of potassium in both interstitial fluid and blood. Although not shown, whether this calibration graph is repeated after several penetrations in animal skin the calibration parameters are maintained.

The same experiment concerning the calibration graph can be accomplished with other microneedles or needles accordingly modified for the transdermal detection of other ions. The potassium microneedles or needles are suitable for the monitoring of fluctuations of potassium levels in interstitial fluid and/or blood while the patch is penetrating the skin, in total analogy to (121). A calibration graph performed before penetration in the skin allows for the conversion of the dynamic potential recorded with the (micro)needle patch penetrating the skin while varying potassium concentration in the interstitial fluid. As a result, a comparison of the recorded potential with the calibration graph provides the transdermal potassium concentration, in total analogy with the potassium-selective (micro)needle patch (121). Analytical performance of every microneedle or needle for ion sensing is to be evaluated to assure the accurate transdermal detection of the analyte concentration in interstitial fluid or blood. The working electrodes (137) and (148) may be modified in order to reach the desired analytical performances. One advantage of (137) and (148) in relation to (121) is a reduced risk of the detachment of all the layers for providing the sensing concept. One advantage of (121) in relation to (137) and (148) is the simplicity of the hand-made manufacturing with a lower skills requirement for the manufacturer. One advantage of the internal modification over external one is that while both provide individual microneedle or needle for each electrode, with the internal modification both the working and the reference electrode can be implemented in the same microneedle or needle by previous compartmentation (142A). In that embodiment the microneedle or needle is first internally modified to provide two different (and made of isolated material if necessary) compartments that are individually modified to work as the working and the reference electrode, respectively.

In one specific example, the (micro)needle patch has a common reference electrode for all the working electrodes of the type 137 or 148. There are three possibilities here: (i) all the electrodes are prepared with the same ion-selective membrane in order to obtain the reproducibility of the transdermal measurements; (ii) every electrode is fabricated with a different ion-selective membrane according to the ion analyte, here it is possible to measure cations and anions simultaneously; and the last option (iii) is a combination if (i) and (ii). In one variation of this example, the modified microneedles or needles are of different lengths to reach the interstitial fluid and the bloodstream by means of the same patch. This patch has applicability for the measurements of the same analyte in both biological fluids aiming at finding correlations with clinical relevance. The (micro)needle patch for transdermal detection of ions is compatible with any electrical module. It is here preferred the implementation of miniaturized PCB circuit boards for the wireless transmission of the measurements to a device for data harvesting, treatment and interpretation during on-body portability.

The (micro)needle patch may also be applied for molecule detection with amperometry using a modified working electrode, pseudoreference electrode and pseudocounter electrode. The layers to fabricate these electrodes are the same already described for the preparation of the working electrode 126, the pseudoreference electrode 127 and the pseudocounter electrode 128. For the working electrode: coating of conducting material, coating of the mediator, coating of the ion-selective membrane. For the pseudoreference electrode: Ag/AgCl coating. For the pseudocounter electrode: carbon coating (or any other material serving as pseudocounter electrode). In the three cases, an external coating is needed for the final sealing, biocompatibility and transdermal insertion of the corresponding microneedle. In addition, in the working electrode, the external coating may act to repel interferences and/or to adjust the analytical performances of the electrode and/or any other purpose. Also, the integration of the sensing elements can be accomplished in the form of monolayers in analogous way as explained above for the different version of microneedles and needles modified to obtain working electrodes for ions and biomolecules.

The inner modification of the hollow microneedle or needle is to be accomplished by any of the two procedures already described for the method 2, i.e., the modification of the wall(s) or external wire(s) to be introduced in the hollow microneedle.

Depending on the length of the microneedle or needle, the electrodes will reach the interstitial fluid (less than 1000 μm) or blood (>1500 μm reaching the bloodstream). The advantages and drawbacks above described for method 2 over method 1 in the case of ion sensing with the working electrode and reference electrode are applicable for the case of molecule sensing based on the working electrode, the pseudoreference electrode and the pseudocounter electrode. All the configurations for the (micro)needle patch described up to now are applicable to molecule determination.

In one variation, the hollow microneedle or needle is compartmented in three spaces (154A) and made of isolating material if necessary. The wall of one space is structured to fabricate the working electrode as per the described steps (154B). The wall in the second space is structured to fabricate the pseudoreference electrode as per the described steps (154C). The wall in the third space is structured to fabricate the pseudocounter electrode as per the described steps (154D). The compartmented hollow microneedle or needle internally modified with the working electrode, the pseudoreference electrode and the pseudocounter electrode is covered by an external coating, e.g. polymeric layer of polyurethane (PU), to ensure transdermal insertion and biocompatibility (154E). In addition, in the working electrode, the external coating may act to repel interferences and to adjust the analytical performances of the electrode.

At least on microneedle 154E is to be implemented in the wearable (micro)needle patch for molecule sensing.

In another variation, the hollow microneedle or needle is compartmented in three spaces (154A) and the wire to be inserted in one space is structured to fabricate the working electrode as per the described steps (154B), the wire to be inserted in the second space is structured to fabricate the pseudoreference electrode as per the described steps (154C) and, the wire to be inserted in the third space is structured to fabricate the pseudocounter electrode as per the described steps (154D). The dimensions of the utilized wires are selected to ensure its implementation in the compartments of the microneedle or needle after modification, as explained above. The compartmented hollow microneedle or needle internally modified with the working electrode, the pseudoreference electrode and the pseudocounter electrode is covered by an external coating, e.g., polymeric layer of polyurethane (PU), to ensure transdermal insertion and biocompatibility (154E). In addition, in the working electrode, the external coating may act to repel interferences and/or to adjust the analytical performances of the electrode and/or any other purpose.

At least on microneedle 154E is to be implemented in the wearable patch for (bio)molecule sensing.

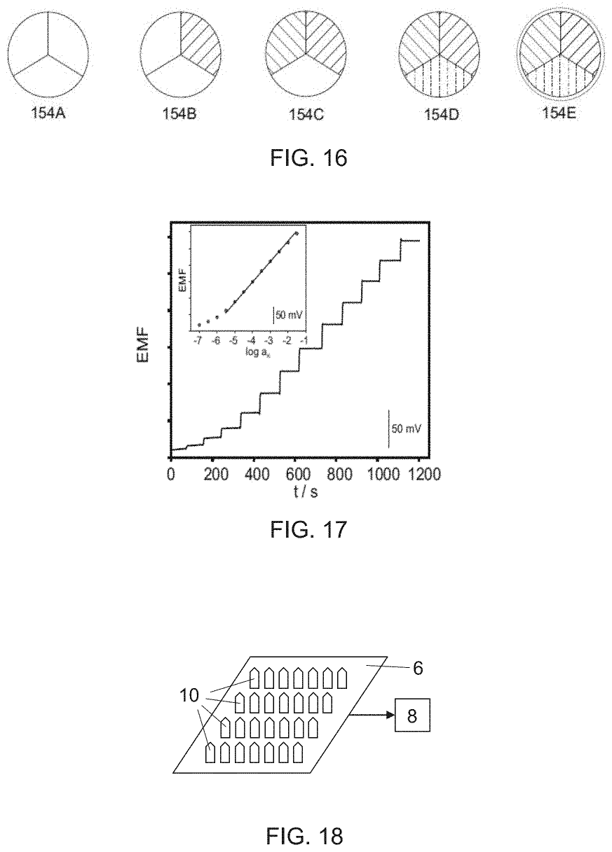

FIG. 16 discloses:
154A: cross-sectional view of compartmented hollow microneedle or needle.
154B: cross-sectional view of compartmented hollow microneedle or needle modified with the working electrode
154C: cross-sectional view of compartmented hollow microneedle or needle modified with the working electrode and the pseudoreference electrode.
154D: cross-sectional view of compartmented hollow microneedle or needle modified with the working electrode, the pseudoreference electrode and the pseudocounter electrode.
154E: cross-sectional view of compartmented hollow microneedle or needle modified with the working electrode, the pseudoreference electrode and the pseudocounter electrode, view from the top of the hole after the deposition of the external layer (e.g., PU).

Below is described using the (micro)needle patch for molecule detection.

In one specific example, the wearable (micro)needle patch comprises a working electrode prepared for glucose detection, the pseduroreference electrode, and the pseudocounter electrode. The glucose sensor comprises a layer of iron hexacyanoferrate (Preussian blue) as the mediator, glucose oxidase as the enzyme layer and Nafion as the external coating. The psedudoreference electrode and the pseudocounter electrode were prepared as above described. The preparation of the working electrode may be modified in order to provide a linear calibration graph comprising the expected levels of glucose in both interstitial fluid and blood. Although not shown, whether this calibration graph is repeated after several penetrations in animal skin the calibration parameters are maintained. The calibration graph can be analogously accomplished with other microneedles or needles accordingly modified for the transdermal detection of other molecules. The working electrode fabrication can be modified in order to shown suitability for the monitoring of fluctuations of molecules levels in interstitial fluid and blood while the patch is penetrating the skin. A calibration graph performed before penetration in the skin allows for the conversion of the dynamic potential recorded with the (micro)needle patch penetrating the skin while varying molecule concentration in the interstitial fluid or blood. As a result, a comparison of the recorded potential with the calibration graph provides the transdermal molecule concentration. One advantage of the internal modification over external modification is that while both provide individual (micro)needle for each electrode, with the internal modification the working electrode, the pseudoreference electrode, and the pseudocounter electrode can be implemented in the same microneedle or needle. The microneedle or needle is first internally modified to provide different compartments that are individually modified to function as the working and as the reference electrode.

The (micro)needle patch for transdermal molecule detection may incorporate as many working/pseudoreference/pseudocounter electrodes as desired. Multi-molecule transdermal detection in amperometric mode is different than the multi-ion transdermal detection in potentiometric mode. For multi-molecule detection, every working electrode has its own pseudoreference and pseudocounter electrodes. The limiting factor here is that the patch becomes bigger with more bunches of electrodes. The user decides the compromise between the final size of the patch and the number of analytes to be detected. There are three possibilities here: (i) all the working electrodes are prepared for the same analyte in order to obtain the reproducibility of the transdermal measurements; (ii) every electrode is fabricated for a different analyte; and the last option (iii) is a combination if (i) and (ii).

In another variant, the (micro)needle patch may combine both ion and molecule detection with potentiometry and amperometry mode, respectively. In one specific example, the modified microneedles or needles are of different lengths to reach the interstitial fluid and the bloodstream by means of the same patch. This patch has applicability for the measurements of the same analyte in both biological fluids aiming at finding correlations with clinical relevance. The (micro)needle patches for transdermal detection of molecules and molecules/ions are compatible with any electrical connections. It is here of interest the implementation of miniaturized PCB circuit boards for the wireless transmission of the measurements to a device for data harvesting, treatment and interpretation.

The present invention is not limited to the above-described preferred embodiments. Various alternatives, modifications and equivalents may be used. Therefore, the above embodiments should not be taken as limiting the scope of the invention, which is defined by the appending claims.

1. A method of modifying the external surfaces of at least two solid microneedles or needles to be arranged in a wearable patch configured for ion and/or molecule on-body transdermal sensing, the method comprises:
providing at least one microneedle or needle structured to function as a working electrode for ion sensing or (bio)molecule sensing, coating said at least one microneedle or needle with a coating to improve the conductivity of the microneedle or needle, and attaching the at least one microneedle or needle to a substrate of said patch, and
providing at least one microneedle or needle structured to function as a reference electrode for ion sensing, or as a pseudoreference electrode for (bio)molecule sensing, coating said at least one microneedle or needle with an Ag/AgCl layer coating to improve the conductivity of the microneedle or needle and additionally serving as a reference electrode or pseudoreference electrode, and attaching the at least one microneedle or needle to the substrate of said patch.

2. The method according to claim 1, comprising providing at least one solid microneedle or needle structured to function as a pseudocounter electrode for molecule sensing, coating said at least one microneedle or needle with a coating to improve the conductivity of the microneedle and providing a constant long-term electrochemical potential and thereby serving as a base material of the pseudocounter electrode, and attaching the at least one microneedle or needle to the substrate of said patch, and coating the coating to improve the conductivity with an external polymeric film, e.g. a polyurethane (PU) film.

3. The method according to claim 1, wherein the method of coating said working electrode for ion sensing comprises coating the coating to improve the conductivity with a ion-to-electron transducer layer, and coating the ion-to-electron transducer layer with an ion-selective membrane, and wherein the method of coating said working electrode for (bio)molecule sensing comprises coating the coating to improve the conductivity with a mediator layer, and coating the mediator layer with an enzyme film, and coating the enzyme layer with an additional external film for different purposes.

4. The method according to claim 1, wherein the method of coating said reference electrode for ion sensing comprises coating the coating to improve the conductivity with a reference membrane film, and coating the reference membrane film with an external polymeric film, e.g. a polyurethane (PU) film, and wherein the method of coating said pseudoreference electrode for (bio)molecule sensing comprises coating the coating to improve the conductivity with an external polymeric film, e.g. a polyurethane (PU) film.

5. A wearable patch configured for ion and/or (bio)molecule on-body transdermal sensing, comprising at least two microneedles or needles modified according to claim 1, wherein the patch is configured to be applied for any electrochemical technique, e.g., potentiometry and amperometry, and for any analyte, and wherein the patch is configured to be connected to a device adapted to process sensed electrochemical values.

6. A method of modifying the surfaces of inner walls of at least one hollow microneedle or needle, or the surfaces of at least one conductive wire to be introduced in the hollow microneedle or needle, wherein each of these two internal modifications is implemented in individual microneedles, or needles, or is in internally compartmented microneedles or needles, said at least one microneedle or needle is to be arranged in a wearable patch configured for ion and/or (bio)molecule on-body transdermal sensing, the method comprises:
providing a surface of the inner wall of a hollow (compartmented) microneedle or needle, or a surface of a conductive wire to be introduced into a hollow microneedle or needle or in one compartment inside a hollow microneedle or needle, in both cases, the surface is structured to function as a working electrode for ion sensing or (bio)molecule sensing, coating said surface with a coating to improve the conductivity of the microneedle/wire, and attaching the microneedle to a substrate of said patch, and
providing a surface of the inner wall of a (compartmented) hollow microneedle or needle, or a surface of a conductive wire to be introduced into a hollow microneedle or needle or in one compartment inside a hollow microneedle or needle, the surface is structured to function as a reference electrode for ion sensing, or as a pseudoreference electrode for (bio)molecule sensing, coating said surface with an Ag/AgCl layer coating to improve the conductivity of the microneedle or needle and additionally serving as a reference electrode or pseudoreference electrode, and attaching the microneedle or needle to the substrate of said patch.

7. The method according to claim 6, comprising providing a inner surface of a (compartmented) hollow microneedle or needle, or a surface of a conductive wire to be introduced inside a hollow microneedle or needle, with the surface structured to function as a pseudocounter electrode for (bio)molecule sensing, coating said surface with a coating to improve the conductivity of the microneedle or needle and providing a constant long-term electrochemical potential and thereby serving as a base material of the pseudocounter electrode, and attaching the least one microneedle or needle to the substrate of said patch, and coating the coating to improve the conductivity with an external polymeric film.

8. The method according to claim 6, wherein the method of coating said surface to function as working electrode for ion sensing comprises coating the coating to improve the conductivity with a ion-to-electron transducer layer, and coating the ion-to-electron transducer layer with an ion-selective membrane, and wherein the method of coating said surface to function as working electrode for molecule sensing comprises coating the coating to improve the conductivity with a mediator layer, and coating the mediator layer with an enzyme film, and coating the enzyme layer with an additional external film for different purposes.

9. The method according to claim 6, wherein the method of coating said surface to function as reference electrode for ion sensing comprises coating the coating to improve the conductivity with a reference membrane film, and coating the reference membrane film with an external polymeric film, e.g. a polyurethane (PU) film, and wherein the method of coating said surface to function as pseudoreference electrode for (bio)molecule sensing comprises coating the coating to improve the conductivity with an external polymeric film.

10. A wearable patch configured for ion and/or (bio)molecule on-body transdermal sensing, comprising at least one microneedle or needle modified according to claim 6, wherein the microneedle patch is configured to be applied for any electrochemical technique, and for any analyte, and wherein the patch is configured to be connected to a device adapted to process sensed electrochemical values.

11. The method according to claim 1, of modifying the external surfaces of at least two solid microneedles or needles to be arranged in a wearable patch configured for the potentiometric detection of ions through on-body transdermal and painless sensing in interstitial fluid and/or blood, wherein the method comprises:
providing at least one microneedle or needle chemically and/or physically structured to function as a working electrode selective for one ion, attaching the at least one microneedle or needle to a polymeric substrate and covering the (micro)needle-substrate architecture for the working electrode for ion detection with a polymer-based coating for sealing and avoiding detachment before, during and after skin penetration as well as providing biocompatibility, and
providing at least one microneedle or needle structured to function as a reference electrode, coating said at least one microneedle or needle with an Ag/AgCl layer, depositing a reference membrane, attaching the at least one microneedle or needle to the same substrate of said working electrode and covering the (micro)needle-substrate architecture for the reference electrode with a polymer-based coating for sealing and avoiding detachment before, during and after skin penetration as well as providing biocompatibility, wherein the method of preparing said working electrode for ion sensing comprises coating the microneedle, needle or wire to improve the conductivity or a direct modification of the microneedle, needle or wire, depending on the former material, and adding next the ion-to-electron transducer layer by either a chemical or physical procedure, and adding then an ion-selective membrane by chemical and/or physical immobilization of each component (polymeric core, ion-exchanger and ionophore).

12. The method according to claim 1, of modifying the external surfaces of at least three solid microneedles or needles to be arranged in a wearable patch configured for the amperometric detection of (bio)molecules through on-body transdermal and painless sensing in interstitial fluid and/or blood, wherein the method comprises:
providing at least one microneedle or needle chemically and/or physically structured to function as a working electrode selective for one (bio)molecule, attaching the at least one microneedle or needle to a polymeric substrate and covering the (micro)needle-substrate architecture for the working electrode for (bio)molecule detection with a polymer-based coating for sealing and avoiding detachment before, during and after skin penetration as well as providing biocompatibility, and
providing at least one microneedle or needle structured to function as a pseudoreference electrode, coating said at least one microneedle or needle with a Ag/AgCl layer, attaching the at least one microneedle or needle to the same substrate of said working electrode for (bio)molecule and covering the (micro)needle-substrate architecture for the pseudoreference electrode with a polymer-based coating for sealing and avoiding detachment before, during and after skin penetration as well as providing biocompatibility.
providing at least one microneedle or needle structured to function as a pseudocounter electrode, coating said at least one microneedle or needle with a carbon layer, attaching the at least one microneedle or needle to the same substrate of said working electrode for (bio)molecule and the said pseudoreference electrode and covering the (micro)needle-substrate architecture for the pseudocounter electrode with a polymer-based coating for sealing and avoiding detachment before, during and after skin penetration as well as providing biocompatibility, wherein the method of preparing said working electrode for (bio)molecule sensing comprises coating the microneedle, needle or wire to improve the conductivity or a direct modification of the microneedle, needle or wire, depending on the former material, and adding next a redox mediator layer by either a chemical or physical procedure, and immobilizing then different enzymes by either a chemical or physical procedure, and selecting the external polymeric layer according to different functions (together with the sealing of the (micro)needle-substrate architecture) to control the analytical performance of the (bio)molecule sensor.

13. The method according to claim 6, of internally modifying hollow microneedles or needles with at least one conductive wire that is introduced in the (micro)needle cavity at different distances of its entire length to provide either direct contact with the sample at the (micro)needle tip or and internal cavity in the (micro)needle to host the sample, and said at least one wire is configured for potentiometric readout by a working electrode for selective ion sensing and a reference electrode that are jointly arranged in a wearable patch for on-body transdermal and painless sensing in interstitial fluid and/or blood, wherein the method comprises:
providing at least one conductive wire chemically and/or physically structured to function as a working electrode selective for one ion, introducing the wire into the hollow microneedle or needle at the desired distance from the tip, attaching the at least one microneedle or needle containing the working electrode wire to a polymeric substrate, and
providing at least one conductive wire chemically and/or physically structured to function as a reference electrode, coating said at least one wire with an Ag/AgCl layer, depositing a reference membrane, introducing the wire into the hollow microneedle or needle at the desired distance from the tip, attaching the at least one microneedle or needle containing the reference electrode wire to a polymeric substrate together with the working electrode for ion sensing, wherein the method of preparing said working electrode for ion sensing comprises coating the microneedle, needle or wire to improve the conductivity or a direct modification of the microneedle, needle or wire, depending on the former material, and adding next the ion-to-electron transducer layer by either a chemical or physical procedure, and adding then an ion-selective membrane by chemical and/or physical immobilization of each component (polymeric core, ion-exchanger and ionophore).

14. The method according to claim 6, of internally modifying hollow microneedles or needles with at least one conductive wire that is introduced in the (micro)needle cavity at different distances of its entire length to provide either direct contact with the sample at the (micro)needle tip or and internal cavity in the (micro)needle to host the sample, and said at least one wire is configured for amperometric readout by a working electrode for (bio)molecules, a pseudoreference electrode and a pseudocounter electrode that are jointly arranged in a wearable patch for on-body transdermal and painless sensing in interstitial fluid and/or blood, wherein the method comprises:
providing at least one conductive wire chemically and/or physically structured to function as a working electrode for (bio)molecules, introducing the wire into the hollow microneedle or needle at the desired distance from the tip, attaching the at least one microneedle or needle to a polymeric substrate, and
providing at least one conductive wire chemically and/or physically structured to function as a pseudoreference electrode, coating said at least one wire with an Ag/AgCl layer and adding a polymeric external layer, introducing the wire into the hollow microneedle or needle at the desired distance from the tip, attaching the at least one microneedle or needle to a polymeric substrate together with the working electrode for (bio)molecule sensing, and
providing at least one conductive wire chemically and/or physically structured to function as a pseudocounter electrode, coating said at least one wire with carbon layer and adding a polymeric external layer, introducing the wire into the hollow microneedle or needle at the desired distance from the tip, attaching the at least one microneedle or needle to a polymeric substrate together with the working electrode for (bio)molecule sensing, and the pseudoreference electrode, wherein the method of preparing said working electrode for (bio)molecule sensing comprises coating the microneedle, needle or wire to improve the conductivity or a direct modification of the microneedle, needle or wire, depending on the former material, and adding next a redox mediator layer by either a chemical or physical procedure, and immobilizing then different enzymes by either a chemical or physical procedure, and selecting the external polymeric layer according to different functions (together with the sealing of the (micro)needle-substrate architecture) to control the analytical performance of the (bio)molecule sensor.

15. The method according to claim 6, of internally modifying hollow microneedles or needles through chemical and/or physical modification of the internal walls to provide at least two hollow microneedles or needles to be arranged in a wearable patch configured for the potentiometric detection of ions through on-body transdermal and painless sensing in interstitial fluid and/or blood, wherein the method comprises:
providing at least one microneedle or needle chemically and/or physically structured to function as a working electrode selective for one ion, attaching the at least one microneedle or needle to a polymeric substrate, and
providing at least one microneedle or needle structured to function as a reference electrode, coating said at least one microneedle or needle with an Ag/AgCl layer, depositing a reference membrane, depositing an external polymeric layer, attaching the at least one microneedle or needle to the same substrate of said working electrode, wherein the method of preparing said working electrode for ion sensing comprises coating the microneedle, needle or wire to improve the conductivity or a direct modification of the microneedle, needle or wire, depending on the former material, and adding next the ion-to-electron transducer layer by either a chemical or physical procedure, and adding then an ion-selective membrane by chemical and/or physical immobilization of each component (polymeric core, ion-exchanger and ionophore).

16. The method according to claim 6, of internally modifying hollow microneedles or needles through chemical and/or physical modification of the internal walls to provide at least three hollow microneedles or needles to be arranged in a wearable patch configured for the amperometric detection of (bio)molecules through on-body transdermal and painless sensing in interstitial fluid and/or blood, wherein the method comprises:
providing at least one microneedle or needle chemically and/or physically structured to function as a working electrode selective for one (bio)molecule, attaching the at least one microneedle or needle to a polymeric substrate, and
providing at least one microneedle or needle structured to function as a pseudoreference electrode, coating said at least one microneedle or needle with a Ag/AgCl, adding a polymeric layer, attaching the at least one microneedle or needle to the same substrate of said working electrode for (bio)molecule, and
providing at least one microneedle or needle structured to function as a pseudocounter electrode, coating said at least one microneedle or needle with a carbon layer, adding a polymeric coating, attaching the at least one microneedle or needle to the same substrate of said working electrode for (bio)molecule and the said pseudoreference electrode, wherein the method of preparing said working electrode for (bio)molecule sensing comprises coating the microneedle, needle or wire to improve the conductivity or a direct modification of the microneedle, needle or wire, depending on the former material, and adding next a redox mediator layer by either a chemical or physical procedure, and immobilizing then different enzymes by either a chemical or physical procedure, and selecting the external polymeric layer according to different functions (together with the sealing of the (micro)needle-substrate architecture) to control the analytical performance of the (bio)molecule sensor.

17. (canceled)

18. (canceled)

19. (canceled)

20. (canceled)

21. (canceled)
